(19) United States
(12) Patent Application Publication
(10) Pub. No.: US 2026/0345450 A1
(43) Pub. Date: Nov. 5, 2026

(54) COSMETIC

(71) Applicant: SHISEIDO COMPANY, LTD., Tokyo (JP)

(72) Inventors: Yusuke OKUYAMA, Tokyo (JP); Yudai MATSUZAKI, Tokyo (JP)

(73) Assignee: SHISEIDO COMPANY, LTD., Tokyo (JP)

(21) Appl. No.: 19/545,007

(22) Filed: Feb. 19, 2026

(30) Foreign Application Priority Data
Mar. 4, 2025 (JP) 2025-033677

(51) Int. Cl. A61K 8/34; A61K 8/81; A61Q 19/02

(52) U.S. Cl. CPC A61K 8/347; A61K 8/8188; A61Q 19/02

(57) ABSTRACT

The present invention provides a cosmetic having excellent feel during use, which is non-sticky, blends well into skin, and can impart moist feeling at the time of use. The cosmetic according to the present invention comprises (A) a bakuchiol; and (B) a polymer having a structural unit derived from acrylamide alkylsulfonic acid or a salt thereof.

### CROSS-REFERENCE TO RELATED APPLICATIONS

### Background of the Invention

### Field of the Invention

The present invention relates to a cosmetic that is less sticky when applied to skin and is excellent in feel during use.

### Background Art

A thickener may be blended for the purpose of imparting an appropriate viscosity to cosmetics. As such thickeners, various thickeners have been studied, and among them, polymers having a structure derived from acrylamide alkylsulfonic acid can impart acid resistance or salt resistance to cosmetics, and are blended into various cosmetics. However, cosmetics or the like in which such polymers are blended may have, for example, an insufficient feel during use, such as a sticky feeling, insufficient blending into skin, or inferior moist feeling at the time of use, when using the cosmetics. For this reason, there has been a demand for development of a cosmetic excellent in feel during use while maintaining the effect provided by a polymer having a structure derived from acrylamide alkylsulfonic acid.

On the other hand, various agents are blended in cosmetics or the like in order to improve the skin condition. As such agents, various agents have been studied, and for example, use of retinol as an agent having a whitening effect or a bakuchiol having an effect similar thereto has been studied.

### CITATION LIST

### Patent Literature

Patent Literature 1: JP 2022-079822 A

### SUMMARY OF THE INVENTION

According to the present invention, there is provided a cosmetic having excellent feel during use, which is non-sticky, blends well into skin, and can impart moist feeling at the time of use.

According to the present invention, the following invention is provided.

[1]

A cosmetic including:
(A) a bakuchiol; and
(B) a polymer having a structural unit derived from acrylamide alkylsulfonic acid or a salt thereof.
   [2]

The cosmetic according to [1], in which the structural unit is represented by Formula (b):

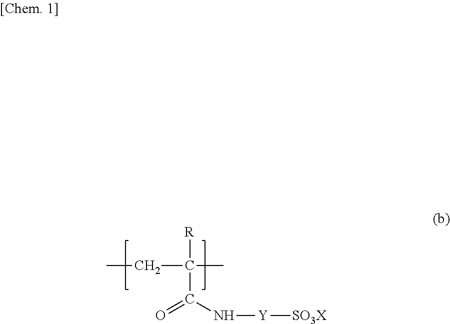

(in the formula,
R is a hydrogen atom, methyl, or ethyl,
Y is an alkylene group having 1 to 9 carbon atoms, and
X is an ammonium, alkali metal, or alkaline earth metal ion).
   [3]

The cosmetic according to [1] or [2], in which component (B) comprises a polymer selected from the group consisting of (sodium acrylate/sodium acryloyldimethyltaurate) copolymer, (ammonium acryloyldimethyltaurate/beheneth-25 methacrylate) crosspolymer, and (dimethylacrylamide/sodium acryloyldimethyltaurate) crosspolymer.

[4]

The cosmetic according to any one of [1] to [3], further including: (C) an ester oil.

[5]

The cosmetic according to [4], in which component (C) comprises (c1) an ester oil derived from 2-ethylhexanoic acid.

[6]

The cosmetic according to any one of [1] to [5], further including: (D) a polyglycerin fatty acid ester.

[7]

The cosmetic according to any one of [1] to [6], in which a ratio B/A of a content amount of component (B) to a content amount of component (A) contained in the cosmetic is 0.01 to 40.

[8]

The cosmetic according to any one of [4] to [7], in which a ratio C/A of a content amount of component (C) to a content amount of component (A) contained in the cosmetic is 0.01 to 200.

[9]

The cosmetic according to any one of [1] to [8], which is an oil-in-water cosmetic.

According to the present invention, a cosmetic excellent in feel during use is provided. More specifically, the cosmetic can exhibit properties of being non-sticky during use, blending well into skin, and imparting a moist feeling at the time of use.

### DETAILED DESCRIPTION OF THE INVENTION

A cosmetic according to the present invention (hereinafter, sometimes referred to simply as a “cosmetic”) comprises (A) a bakuchiol; and (B) a polymer having a structural unit derived from acrylamide alkylsulfonic acid or a salt thereof. Each of these components and components that can be additionally blended in will be described below.

### (A) Bakuchiol

The cosmetic according to the present invention contains a bakuchiol (hereinafter, sometimes referred to as component (A)). The bakuchiol is a terpenophenol compound represented by the following Formula (a):

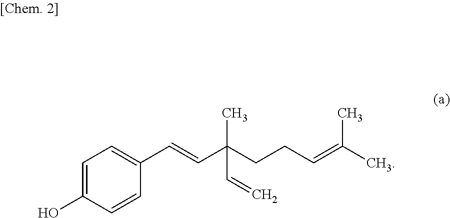 [SMILES: C=CC(C)(/C=C/c1ccc(O)cc1)CCC=C(C)C]

The bakuchiol can be obtained from a natural product or can be prepared by synthesis, and a production method is not particularly limited, and can be obtained, for example, by extraction from babchi seed. It is known that the bakuchiol has an antioxidant action and an anti-inflammatory action, and exhibits an effect similar to that of retinol conventionally used as a whitening agent. The bakuchiol that can be used in the present invention includes derivatives and salts thereof in addition to the bakuchiol itself.

In the present invention, component (A) can also obtain a whitening effect peculiar to bakuchiol, and also has an unexpected effect that the feel during use can be improved, when combined with polyglycerin described later. Although the mechanism by which these effects are obtained is not clear, component (A) exhibits an effect of significantly reducing the deterioration of the feel during use caused by polyglycerin.

In the present invention, a content of component (A) can be adjusted according to a content of component (B) described later, and is, for example, preferably 0.001 to 3 mass %, and more preferably 0.01 to 2 mass %, based on a total mass of the cosmetic.

(B) Polymer Having Structural Unit Derived from Acrylamide Alkylsulfonic Acid or Salt Thereof

The cosmetic according to the present invention comprises a polymer having a structural unit derived from acrylamide alkylsulfonic acid or a salt thereof (hereinafter, sometimes referred to as component (B)). As a salt of acrylamide alkylsulfonic acid, for example, a metal salt such as an alkali metal or an alkaline earth metal, or an organic amine salt such as ammonia, trimethylamine, or triethanolamine can be used.

A structural unit derived from acrylamide alkylsulfonic acid or a salt thereof is preferably, for example, a structural unit represented by Formula (b).

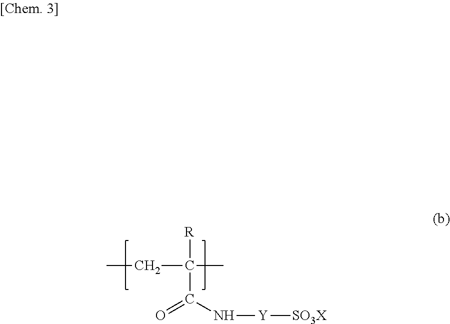

(In the formula,

R is a hydrogen atom, methyl, or ethyl,

Y is an alkylene group having 1 to 9 carbon atoms, and

X is an ammonium, alkali metal, or alkaline earth metal ion.)

Component (B) may be a homopolymer composed only of a structural unit derived from acrylamide alkylsulfonic acid or a salt thereof, or a copolymer containing another structural unit. The other structural unit can be a derivative of a vinyl compound such as acrylic acid or methacrylic acid, acrylamide or methacrylamide, or an ester of acrylic acid or methacrylic acid.

Specifically, examples of component (B) can comprise a polymer selected from the group consisting of
(sodium acrylate/sodium acryloyldimethyltaurate) copolymer,
(ammonium acryloyldimethyltaurate/beheneth-25 methacrylate) crosspolymer, and
(dimethylacrylamide/sodium acryloyldimethyltaurate) crosspolymer. In the present invention, component (B) can be used in combination of two or more kinds of polymers, and in this case, component (B) preferably contains the polymers specifically described herein.

In the present invention, the content of component (B) is preferably 0.001 to 3 mass %, and more preferably 0.05 to 2 mass %, based on the total mass, in order to favorably maintain the feel during use when the cosmetic is used.

In addition, in the present invention, it is preferable to consider the balance of the content amounts of components (A) and (B) in order to obtain feel during use that is low in sticky feeling and high in moist feeling when the cosmetic is used. Specifically, a mass ratio B/A of the content amount of component (B) to the content amount of component (A) contained in the cosmetic is preferably 0.01 to 40, and more preferably 0.05 to 30.

### (C) Ester Oil

The cosmetic according to the present invention comprises the above-described components (A) and (B) as essential components, but may further contain other components as necessary. One of these other components is an ester oil (hereinafter, referred to as component (C)).

Component (C) further suppresses sticky feeling when the cosmetic according to the present invention is used, and exhibits an effect of improving moist feeling.

When the cosmetic is an emulsified cosmetic, component (C) dissolves an oil-soluble component among the components contained in the cosmetic to form an oil phase in the cosmetic. When the cosmetic is an oily cosmetic, component (C) functions as a solvent or the like.

As component (C), any ester oil used in cosmetics and cleansers can be selected and used. Examples thereof include
ester oils such as
pentaerythrityl tetraethylhexanoate,
cetyl ethylhexanoate,
triethylhexanoin,
pentaerythrityl tetra(behenate/benzoate/ethylhexanoate),
diisopropyl sebacate,
jojoba oil,
di(phytosteryl/octyldodecyl) lauroyl glutamate,
triisostearin,
glyceryl diisostearate,
(phytosteryl/behenyl) dimer dilinoleate,
(phytosteryl/isostearyl/cetyl/stearyl/isostearyl/cetyl/stearyl/behenyl) dimer dilinoleate,
isopropyl palmitate,
phytosteryl macadamiate,
ethylhexyl palmitate,
myristyl myristate,
isopropyl myristate,
tripropylene glycol dipivalate, and
isodecyl neopentanoate.

Among these, ester oils derived from 2-ethylhexanoic acid, specifically pentaerythrityl tetraethylhexanoate, cetyl ethylhexanoate, and triethylhexanoin are preferable. In the present invention, component (C) can be used in combination of two or more oils, and in this case component (C) preferably contains the polymer specifically described herein, and particularly preferably contains an ester oil derived from 2-ethylhexanoic acid.

In the present invention, the content of component (C) is preferably 0.05 to 10 mass %, and more preferably 0.1 to 8 mass %, based on the total mass, in order to realize a feel during use when the cosmetic is used, particularly a suppressed sticky feeling and improved moist feeling.

In addition, in the present invention, it is preferable to consider the balance of the content amounts of components (A) and (C) in order to obtain feel during use that is low in sticky feeling and high in moist feeling when the cosmetic is used. Specifically, a mass ratio C/A of the content amount of component (C) to the content amount of component (A) contained in the cosmetic is preferably 0.01 to 200, and more preferably 0.1 to 150.

### (D) Polyglycerin Fatty Acid Ester

The cosmetic according to the present invention can further comprise a polyglycerin fatty acid ester (hereinafter referred to as component (D)). In the present invention, component (D) functions as a surfactant. Component (D) can be prepared, for example, by esterifying fatty acid and polyglycerin. Here, in component (D), a portion derived from a fatty acid may be referred to as a fatty acid moiety, and a portion derived from polyglycerin may be referred to as a polyglycerin moiety. The fatty acid moiety has lipophilicity, and the polyglycerin moiety has hydrophilicity. While the hydrophilicity of the polyglycerin fatty acid ester itself changes depending on the balance between the fatty acid moiety and the polyglycerin moiety, in the present invention, component (D) having appropriate hydrophilicity can be selected depending on the component constituting the cosmetic. Therefore, HLB of component (D) is not particularly limited, and is preferably 3 to 18. In an embodiment of the present invention, hydrophilicity is preferably high, and for example, HLB is preferably 10 or higher.

The number of carbon atoms in the fatty acid-derived moiety of the polyglycerin fatty acid ester is preferably 10 to 18, and more preferably 12 to 16. The fatty acid may be a saturated fatty acid or an unsaturated fatty acid, and may be a linear fatty acid or a branched fatty acid. Examples of such a fatty acid include lauric acid, myristic acid, palmitic acid, oleic acid, stearic acid, and isostearic acid, and lauric acid is particularly preferable. The polyglycerin fatty acid ester may comprise 1 to 3 fatty acid moieties and can be polyglycerin monofatty acid ester, polyglycerin difatty acid ester, or polyglycerin trifatty acid ester.

Examples of the polyglycerin fatty acid ester include polyglyceryl-10 laurate, polyglyceryl-6 laurate, polyglyceryl-4 laurate, polyglyceryl-2 laurate, polyglyceryl-10 myristate, polyglyceryl-10 palmitate, polyglyceryl-6 stearate, polyglyceryl-10 isostearate, polyglyceryl-2 diisostearate, polyglyceryl-6 dicaprate, polyglyceryl-10 oleate, and polyglyceryl-6 oleate. Among them, highly hydrophilic ones such as polyglyceryl-10 laurate, polyglyceryl-6 laurate, polyglyceryl-4 laurate, polyglyceryl-10 myristate, and polyglyceryl-10 isostearate are preferably used.

In the present invention, component (D) can be used in combination of two or more oils, and in this case, component (D) preferably contains a highly hydrophilic oil, specifically, an oil having an HLB of 10 or higher, and an average HLB of entire component (D) is preferably 10 or higher.

In the present invention, the content of component (D) is preferably 0.001 to 5 mass %, and more preferably 0.01 to 3 mass %, based on the total mass, in order to realize a feel during use when the cosmetic is used, particularly a suppressed sticky feeling and improved moist feeling.

The cosmetic according to the invention can further comprise water. As the water, water used for cosmetic products, quasi-drugs, and the like can be used. For example, purified water, ion-exchanged water, and tap water can be used.

The cosmetic according to the present invention can also comprise an oil component other than component (C). Examples of the oil component include hydrocarbon oil, higher fatty acid, higher alcohol, and silicone oil.

Examples of the hydrocarbon oil include isododecane, isohexadecane, isoparaffin, mineral oil (liquid paraffin), paraffin, ceresin, and squalene.

Examples of the higher fatty acid include carboxylic acids having 10 or more carbon atoms.

Examples of the higher alcohol include a linear alcohol and a branched alcohol.

Examples of the silicone oil include chain polysiloxanes such as dimethylpolysiloxane, methylphenylpolysiloxane, and diphenylpolysiloxane, and cyclic polysiloxanes such as octamethylcyclotetrasiloxane, decamethylcyclopentasiloxane, and dodecamethylcyclohexasiloxane. In addition, silicone oil having a vinyl group at the terminal or having a Si—H group can also be used.

The cosmetic according to the present invention can take any form such as an aqueous cosmetic, an oily cosmetic, an oil-in-water cosmetic, or a water-in-oil cosmetic by appropriately containing water or an oil component. Among them, the present invention is preferably an oil-in-water cosmetic.

In addition to the above components, the cosmetic according to the present invention may contain other components usually used for cosmetic products and pharmaceuticals. Examples of such components include the following components, and one kind or two or more kinds can be blended as long as the effects of the present invention are exhibited.

When the cosmetic according to the present invention is an emulsified cosmetic, a surfactant can be blended as a component for stabilizing a dispersion state thereof. The surfactant can be used by being arbitrarily selected from nonionic surfactants, anionic surfactants, cationic surfactants, and amphoteric surfactants other than component (D), which are usually used for external preparations such as cosmetics and quasi-drugs.

The cosmetic according to the present invention contains polyglycerin exhibiting a moisturizing effect, and can contain polyhydric alcohols such as polyethylene glycol, propylene glycol, dipropylene glycol (DPG), and 1,3-butylene glycol (BG) as other moisturizing agents.

In addition, a lower alcohol, a thickener, a sequestering agent, a neutralizing agent, a pH adjusting agent, various agents, an ultraviolet absorber, a powder component, a fragrance, and the like can also be appropriately blended as components.

Examples of the cosmetic according to the present invention include skin care cosmetics (for example, lotion, emulsion, cream, serum, pack, mask, and the like), makeup cosmetics (for example, foundation, makeup base, and the like), skin cleansers (for example, facial cleanser, makeup remover, and the like), sunscreen cosmetics, and ointments. These forms are merely examples, and the cosmetic according to the present invention is not limited to these forms.

### EXAMPLES

The present invention will be specifically described based on the following examples, but the present invention is not limited to these examples. The content is represented in mass % with respect to the total amount, unless otherwise specified.

### Examples 1 to 13, and Comparative Example 1

Cosmetics of examples and comparative example were prepared with the formulation shown in Table 1. Furthermore, regarding the performance of these cosmetics, evaluation was performed as follows.

### [Evaluation of Usability: Stickiness]

The cosmetic prepared above was applied to the skin by ten expert panelists and stickiness during application was evaluated. The evaluation criteria are as follows.

A+: Ten panelists evaluated as having no stickiness.

A: Eight to nine panelists evaluated as having no stickiness.

B: Six to seven panelists evaluated as having no stickiness.

C: Four to five panelists evaluated as having no stickiness.

D: Two to three panelists evaluated as having no stickiness.

E: One or fewer panelists evaluated as having no stickiness.

### [Evaluation of Usability: Ease of Blending and Moist Feeling]

Ten expert panelists applied the cosmetic prepared above to the skin and evaluated the ease of blending during application and moist feeling after application of the cosmetic.

Here, the “ease of blending” means that the cosmetic permeates into the skin, fits without discomfort, and provides a feeling that the application action is finished.

A+: Ten panelists evaluated as having good blending/having moist feeling.

A: Eight to nine panelists evaluated as having good blending/having moist feeling.

B: Six to seven panelists evaluated as having good blending/having moist feeling.

C: Four to five panelists evaluated as having good blending/having moist feeling.

D: Two to three panelists evaluated as having good blending/having moist feeling.

E: One or fewer panelists evaluated as having good blending/having moist feeling.

The obtained results are also shown in Table 1 below.

TABLE 1

| | | Com. | | | | | | |
|---|---|---|---|---|---|---|---|---|
| | | Ex. | Example | | | | | |
| | | 1 | 1 | 2 | 3 | 4 | 5 | 6 |
| | Water | balance | balance | balance | balance | balance | balance | balance |
| A | Bakuchiol | — | 0.1 | 0.05 | 1.5 | 0.1 | 0.1 | 0.1 |
| B | (Sodium acrylate/sodium acryloyl- | 0.3 | 0.3 | 0.3 | 0.3 | 0.1 | 1.5 | — |
| | dimethyltaurate) copolymer | | | | | | | |
| | (Ammonium acryloyldimethyltaurate/ | — | — | — | — | — | — | 0.3 |
| | beheneth-25 methacrylate) crosspolymer | | | | | | | |
| | (Dimethylacrylamide/sodium acryloyl- | — | — | — | — | — | — | — |
| | dimethyltaurate) crosspolymer | | | | | | | |
| C | Cetyl ethylhexanoate | — | — | — | — | — | — | — |
| | Triethylhexanoin | — | — | — | — | — | — | — |
| | Pentaerythrityl tetraethylhexanoate | — | — | — | — | — | — | — |
| D | Polyglyceryl-6 laurate | 0.5 | 0.5 | 0.5 | 0.5 | 0.5 | 0.5 | 0.5 |
| | (HLB: approximately 15) | | | | | | | |
| | Polyglyceryl-2 diisostearate | — | — | — | — | — | — | — |
| | (HLB: approximately 4) | | | | | | | |
| | Glycerin | 5 | 5 | 5 | 5 | 5 | 5 | 5 |
| | BG | 5 | 5 | 5 | 5 | 5 | 5 | 5 |
| | Isohexadecane | q.s. | q.s. | q.s. | q.s. | q.s. | q.s. | q.s. |
| | Polysorbate 80 | q.s. | q.s. | q.s. | q.s. | q.s. | q.s. | q.s. |
| | Sorbitan oleate | q.s. | q.s. | q.s. | q.s. | q.s. | q.s. | q.s. |
| | Phenoxyethanol | q.s. | q.s. | q.s. | q.s. | q.s. | q.s. | q.s. |
| | EDTA-3Na | q.s. | q.s. | q.s. | q.s. | q.s. | q.s. | q.s. |
| | Total | 100 | 100 | 100 | 100 | 100 | 100 | 100 |
| | B/A | — | 3 | 6 | 0.2 | 1 | 15 | 3 |
| | C/A | — | — | — | — | — | — | — |
| Evaluation | Sticky feeling | D | A | A | B | A | B | A |
| | Ease of absorption | D | A | A | A | A | A | A |
| | Moist feeling | D | A | B | B | A | B | B |
| | | Example | | | | | | |
| | | 7 | 8 | 9 | 10 | 11 | 12 | 13 |
| | Water | balance | balance | balance | balance | balance | balance | balance |
| A | Bakuchiol | 0.1 | 0.1 | 0.1 | 0.1 | 0.1 | 0.1 | 0.1 |
| B | (Sodium acrylate/sodium acryloyl- | — | 0.3 | 0.3 | 0.3 | 0.3 | 0.3 | 0.3 |
| | dimethyltaurate) copolymer | | | | | | | |
| | (Ammonium acryloyldimethyltaurate/ | — | — | — | — | — | — | — |
| | beheneth-25 methacrylate) crosspolymer | | | | | | | |
| | (Dimethylacrylamide/sodium acryloyl- | 0.3 | — | — | — | — | — | — |
| | dimethyltaurate) crosspolymer | | | | | | | |
| C | Cetyl ethylhexanoate | — | 2 | 0.5 | 7 | — | — | — |
| | Triethylhexanoin | — | — | — | — | 2 | — | — |
| | Pentaerythrityl tetraethylhexanoate | — | — | — | — | — | 2 | — |
| D | Polyglyceryl-6 laurate | 0.5 | 0.5 | 0.5 | 0.5 | 0.5 | 0.5 | — |
| | (HLB: approximately 15) | | | | | | | |
| | Polyglyceryl-2 diisostearate | — | — | — | — | — | — | 0.5 |
| | (HLB: approximately 4) | | | | | | | |
| | Glycerin | 5 | 5 | 5 | 5 | 5 | 5 | 5 |
| | BG | 5 | 5 | 5 | 5 | 5 | 5 | 5 |
| | Isohexadecane | q.s. | q.s. | q.s. | q.s. | q.s. | q.s. | q.s. |
| | Polysorbate 80 | q.s. | q.s. | q.s. | q.s. | q.s. | q.s. | q.s. |
| | Sorbitan oleate | q.s. | q.s. | q.s. | q.s. | q.s. | q.s. | q.s. |
| | Phenoxyethanol | q.s. | q.s. | q.s. | q.s. | q.s. | q.s. | q.s. |
| | EDTA-3Na | q.s. | q.s. | q.s. | q.s. | q.s. | q.s. | q.s. |
| | Total | 100 | 100 | 100 | 100 | 100 | 100 | 100 |
| | B/A | 3 | 3 | 3 | 3 | 3 | 3 | 3 |
| | C/A | — | 20 | 5 | 70 | 20 | 20 | — |
| Evaluation | Sticky feeling | A | A+ | A+ | A+ | A+ | A+ | A |
| | Ease of absorption | A | A | A | A | A | A | A |
| | Moist feeling | B | A+ | A | A+ | A+ | A+ | A |

1. A cosmetic comprising:
(A) a bakuchiol; and
(B) a polymer having a structural unit derived from acrylamide alkylsulfonic acid or a salt thereof.

2. The cosmetic according to claim 1, wherein said structural unit is represented by Formula (b), 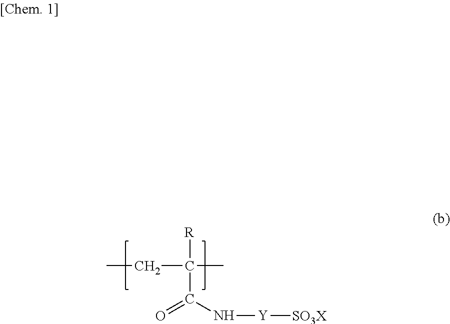
(in the formula,
R is a hydrogen atom, methyl, or ethyl,
Y is an alkylene group having 1 to 9 carbon atoms, and
X is an ammonium, alkali metal, or alkaline earth metal ion).

3. The cosmetic according to claim 1, wherein component (B) comprises a polymer selected from the group consisting of (sodium acrylate/sodium acryloyldimethyltaurate) copolymer, (ammonium acryloyldimethyltaurate/beheneth-25 methacrylate) crosspolymer, and (dimethylacrylamide/sodium acryloyldimethyltaurate) crosspolymer.

4. The cosmetic according to claim 1, further comprising: (C) an ester oil.

5. The cosmetic according to claim 4, wherein component (C) comprises (c1) an ester oil derived from 2-ethylhexanoic acid.

6. The cosmetic according to claim 1, further comprising: (D) a polyglycerin fatty acid ester.

7. The cosmetic according to claim 1, wherein a ratio B/A of a content amount of component (B) to a content amount of component (A) contained in said cosmetic is 0.01 to 40.

8. The cosmetic according to claim 4, wherein a ratio C/A of a content amount of component (C) to a content amount of component (A) contained in said cosmetic is 0.01 to 200.

9. The cosmetic according to claim 1, which is an oil-in-water cosmetic.
